(19) United States
(12) Patent Application Publication
(10) Pub. No.: US 2004/0267568 A1
(43) Pub. Date: Dec. 30, 2004

(54) Creation of a database of biochemical data and methods of use

(76) Inventors: Mark Chandler, Austin, TX (US); Van S. Chandler, Georgetown, TX (US)

Correspondence Address: CONLEY ROSE, P.C., P.O. BOX 684908, AUSTIN, TX, 78768, US

(21) Appl. No.: 10/804,451

(22) Filed: Mar. 19, 2004

Related U.S. Application Data
(62) Division of application No. 09/662,790, filed on Sep. 15, 2000.
(60) Provisional application No. 60/153,941, filed on Sep. 15, 1999.
(60) Provisional application No. 60/227,516, filed on Aug. 24, 2000.

(51) Int. Cl. G06F 17/60; G06F 19/00; G01N 33/48; G01N 33/50; C12P 21/06; G06F 7/00; G06F 17/00

(52) U.S. Cl. 705/2; 702/19; 707/102

(57) ABSTRACT

This invention is based on the observation that all diseases may be diagnosed by analysis of at least about 200-300 biochemicals present in a patient's blood. In addition, disease progression may also be predicted from the profile of biochemicals in the blood. This invention takes advantage of rapid automated methods for determining the concentrations of biochemicals in the blood of a patient and compares these concentrations to a database which includes information derived from a multi-year study of approximately 200,000 persons. From the correlations found between biochemical concentrations in the blood and the existence of disease states, this invention permits the diagnosis of a present disease state in the patient and has the capacity to predict the emergence of future disease states in the patient.

### CROSS-REFERENCE TO RELATED APPLICATIONS

The present application claims the benefit of the priority dates of co-pending provisional Application Ser. No. 60/153,941, filed Sep. 15, 1999 and 60/227,516, filed Aug. 24, 2000, the complete disclosures of which are incorporated by reference herein.

### 1. FIELD OF THE INVENTION

This invention relates to the creation and use of a database comprising biochemical data for a wide range of applications, including diagnosis of disease states, the prognosis for recovery, determination of the onset (or potential therefor) of future disease states, assessment of health or medical condition and the like.

### 2. BACKGROUND OF THE INVENTION

The current approach to medical studies of disease involve the measurement of a few analytes in the blood, exhaustive observation of lifestyle and diet, and occasional experimental control of subject selection by genetic trait or environment. While these measurements can give a vague picture of the elements of a healthy lifestyle, correlation between genetic and environmental factors and a particular disease is usually low. It is believed that genetic variation among individuals is primarily responsible for weak correlation, but the disappointment remains because in spite of billions of dollars spent on medical research, there are very few measurements of analyte, lifestyle, or environment which accurately and consistently predict disease.

U.S. Pat. No. 4,733,354 discloses a method for making a dermatopathological medical diagnosis using a stored database and decision tree analysis.

U.S. Pat. No. 4,874,693 discloses a method for detecting placental dysfunction, which is diagnostic of chromosomal abnormalities through quantifying the hormone human chorionic gonadotropin or its subunits in bodily fluids.

U.S. Pat. No. 5,622,171 discloses a method for diagnosis of a number of breast diseases based on analysis of radiographic images using a computer and a neural network.

U.S. Pat. No. 5,724,983 discloses a method of periodically computing a diagnosis of a patient based on one or more continuous monitored clinical features as detected by an electrocardiograph. A change-in-condition measure is periodically calculated, and an alarm is sounded when a threshold value of the change-in-condition measure is exceeded.

U.S. Pat. No. 5,937,387 discloses a system and method for using a wide variety of factors such as smoking, blood pressure, and dietary cholesterol for an individual patient to compute the physiological age of the patient. This information may be used by the patient to monitor and improve wellness.

Part of our inability to make strong a correlation is our lack of understanding of the function of genes. However, genes can be thought of as the “vocabulary” of biology, while the proteins they express are the “instructions” to biology. Thus, if one could interpret these instructions, then the onset of disease could be detected earlier, and pharmaceuticals could be developed to change the instructions.

To compound the problem, it has been difficult, if not impossible, to use conventional methods to make measurements of a large number of analytes, such as antigens, antibodies, or proteins. This is because conventional methods require obtaining large amounts of test sample. In the case of methods utilizing blood samples, conventional methods require drawing a life-threatening amount of blood. Moreover, the cost of making so many measurements using conventional methods makes such an effort impractical.

Using conventional methods, statisticians would set up controlled, randomized experiments to assign probability distributions in an attempt to associate one or more abnormal protein levels with a disease state. These statisticians would typically find weak correlation, presumably because there are often many different chains of protein interactions, which cause the same disease.

Using causality-inspired methods, the present invention seeks to solve this problem by describing mathematically multiple paths that lead to the same outcome or multiple outcomes off of the same path.

It is thus an object of the present invention, for example, to determine how cancer is triggered in one person by exposure to a particular carcinogen, while cancer is blocked in another person, exposed to the same carcinogen, by the action of one or more proteins (or inaction of one or more defective or “missing” proteins) expressed by one, the other, or both individuals. The differences in protein expression between individuals may be rooted in each individual's unique genetic makeup or exposure to environmental factors.

It is also an object of the present invention to resolve the sometime ambiguous instructions plaguing biology, especially human and animal biology. This object is achieved by first taking measurements, including generating biochemical data from test samples obtained from subjects, to create a computerized model of normal, healthy biology. Then, through the analysis of further test samples obtained from subjects in the midst of a diseased state, the present invention models the chain of protein events that cause the disease to occur.

### 3. SUMMARY OF THE INVENTION

The present invention provides a method of creating a database containing biochemical data from at least about 1,000 subjects, preferably tens of thousands of subjects. The information compiled in the database of the present invention comprises biochemical data generated from one or more test samples obtained from the subjects and can be retrieved or correlated with identifiers of the subjects along with their medical histories. The method comprises (a) providing one or more test samples obtained from one or more subjects; (b) exposing a Multi-Analyte Profile (MAP) Test Panel to at least a portion of the one or more test samples to provide one or more test mixtures, the MAP Test Panel comprising 20 or more subsets of microspheres, the microspheres of one subset being distinguishable from those of another subset and harboring at least one reagent designed to interact selectively, if not specifically, with, and to generate biochemical data concerning, a predetermined analyte; (c) optionally, adding one or more supplemental reagents to the one or more test mixtures to further the generation of the biochemical data; (d) passing the exposed microspheres of the one or more test mixtures through a flow analyzer to extract the biochemical data generated; (e) compiling the biochemical data into a database, which permits retrieval of the biochemical data at least according to the identities or medical histories of the one or more subjects from which the one or more test samples were obtained; and (f) repeating some or all of the foregoing steps until biochemical data from at least about 1,000 subjects are compiled into the database.

Consistent with the objectives of the present invention, a Multi-Analyte Profile (MAP) Test Panel is also provided, which comprises 20 or more subsets of microspheres, the microspheres of one subset being distinguishable from those of another subset and harboring at least one reagent designed to interact selectively, if not specifically, with a predetermined analyte. In preferred embodiments of the invention the MAP Test Panel comprises 50 or more, 75 or more, 100 or more, 200 or more, or 300 or more subsets of microspheres. In a specific embodiment of the invention, the microspheres of one subset are distinguishable from those of another subset by their characteristic fluorescence signatures. Elsewhere in this specification, microspheres having this characteristic fluorescence signature might also be referred to as fluorescence addressable microspheres. The microspheres of the MAP Test Panel typically contain various concentrations of at least two or more fluorescent dyes, sometimes at least three or more fluorescent dyes and, preferably, at least four or more. The at least one reagent comprises any substance that can selectively, if not specifically, interact with an analyte of interest. Typically, the reagent comprises a small molecule, natural product, synthetic polymer, peptide, polypeptide, polysaccharide, lipid, nucleic acid, or combinations thereof. The predetermined analyte can be any of a wide range of substances also. Typically, the predetermined analyte comprises a drug, hormone, antigen, antibody, protein, enzyme, DNA, RNA, or combinations thereof.

Accordingly, the present invention also provides a kit for assaying 20 or more predetermined analytes in a single pass through a flow analyzer comprising a Multi-Analyte Profile (MAP) Test Panel comprising 20 or more subsets of microspheres, the microspheres of one subset being distinguishable from those of another subset and harboring at least one reagent designed to interact selectively, if not specifically, with a predetermined analyte.

It is also an object of the present invention to permit the assessment of a subject's health or medical condition. In a preferred method of conducting such an assessment, one performs the following steps, including: (a) providing one or more test samples obtained from a subject; (b) exposing the one or more test samples to a Multi-Analyte Profile (MAP) Test Panel comprising 20 or more subsets of microspheres, the microspheres of one subset being distinguishable from those of another subset and harboring at least one reagent designed to interact selectively, if not specifically, with a predetermined analyte, which interaction generates biochemical data concerning the predetermined analyte; (c) gathering the biochemical data, if any, generated from the exposure; (d) comparing the biochemical data generated from the one or more samples obtained from the subject with accumulated biochemical data generated from test samples taken periodically from at least about 1,000 individuals over a given time interval, which accumulated biochemical data provide a relationship between one or more predetermined analytes and the health or medical condition of a plurality of individuals whose accumulated biochemical data share similar features; and (e) assessing the health or medical condition of the subject based, at least in part, on the results of the comparison. In a specific embodiment of the method of assessment, the given time interval is as long as about three years, more preferably, as long as about five years or more.

It is also an object of the present invention to provide a method of monitoring the progression or remission of a disease state or a potential for the onset thereof in a subject over a given time interval, which method comprises generating biochemical data from a plurality of test samples obtained from a subject over a given time interval, processing the generated biochemical data to determine one or more features thereof, which one or more features inform of the progression or remission of a disease state or a potential for the onset thereof in the subject.

Similarly, the present invention provides a method of determining the efficacy or consequence(s) of experimental (or established) treatment, e.g., drugs, radiation, surgery, gene or cell therapy, vaccine, diet and the like, by monitoring changes in biochemical data generated from a plurality of test samples obtained from a subject undergoing treatment over a given time interval.

Yet another object of this invention is to diagnose a disease state or future disease state from the concentration profile of about 200-300, preferably more, biochemical analytes in a test sample. Hence, probability and causal relationships between biochemical data and health effects are elucidated.

Still another object of this invention is to provide methods of detecting side effects of drugs by determining the effect of drug administration on the concentration profile of 200-300 biochemical analytes in test samples obtained from subjects receiving drug and control subjects would have not received drug. Of course, biochemical data obtained from the same subjects before and after drug administration can also be utilized These and other objects of the invention will become apparent to the reader upon consideration of the content of this disclosure, including the following description of preferred embodiments.

### 4. DETAILED DESCRIPTION OF THE PREFERRED EMBODIMENTS

Hence, the present invention provides one or more electronic databases comprising biochemical data. A preferred electronic database can be described as comprising an electronically retrievable first set of information derived from a multiplexed analysis of a biological sample of an individual against a Multi-Analyte Profile (MAP) Test Panel comprising a plurality of predetermined analytes and at least an electronically retrievable second set of information which can be correlated with the first set and which is derived from the individual's medical history or medical condition. The first set of information may include quantitative information for each analyte of the MAP Test Panel, which is found in the biological sample. The second set of information may include the individual's phenotypic information and the individual's genetic information. The preferred database of the invention includes the first and second sets of information derived from 1,000 or more, 10,000 or more, 100,000 or more, 200,000 or more, 500,000 or more, 1,000,000 ore more, 10,000,000 or more, or 100,000,000 or more individuals. In the database the correlation includes the individual's medical history or medical condition at the time the biological sample was taken from the individual. In the database the first and the at least second sets of information are gathered at least annually over a period of two or more, three or more, four or more, or five or more years.

The database also includes a relationship amenable to mathematical or computational manipulation comprising (i) one or more rules derived at least in part from a database comprising a first set of information derived from a multiplexed analysis of a biological sample of an individual against a Multi-Analyte Profile (MAP) Test Panel comprising a plurality of predetermined analytes and at least a second set of information which can be correlated with the first set and which is derived from the individual's medical history or medical condition, and (ii) one or more variables dependent at least on input comprising information derived from a multiplexed analysis of a biological sample of the patient against a panel comprising a plurality of predetermined analytes and, optionally, information derived from the patient's medical history or medical condition, which relationship provides information relating to the probability that a patient may be or will be suffering from one or more disease states. This relationship further provides information relating to the prognosis of the patient.

In a specific embodiment of the invention, a database is compiled comprising biochemical data, including the concentrations of biochemicals in blood samples taken from a large number of persons selected to be representative of the population, the blood samples taken annually over a period of at least 5 years. When the blood samples are taken, a medical history is also determined for each person. The concentrations of biochemicals and changes in concentrations of biochemicals are correlated with the medical histories and changes in medical histories of the persons involved. Finally, an algorithm is derived for correlating the concentrations or changes in concentrations of biochemicals in the blood sample with the presence of a disease state or future disease state in the person whose blood is being tested.

The development of the database of this invention will determine the medical relevance of hundreds of biochemical substances found in the blood of thousands of volunteer participants. It will combine this information with in-depth medical histories to provide the clearest picture yet of the complex events that give rise to disease. The validity of this approach has been established by projects like the Framingham Heart Study, but is further supported by two fundamental assumptions. The first is that every clinically relevant biochemical process occurring in the body in some way manifests itself in the blood. The second is that aberrations or perturbations in these processes signal many, if not all, diseases, and that understanding these changes allows the earliest possible detection and most effective treatment of a particular disease.

Currently, the level of biochemical screening proposed for this project can only be performed by technology developed by Luminex Corporation and disclosed as published patent applications: Microparticles with Multiple Fluorescent Signals, WO99/37814; Multiplexed Analysis of Clinical Specimens Apparatus and Methods, WO99/36564; Interlaced Lasers for Multiple Fluorescence Measurement, WO98/59233; and Precision Fluorescently Dyed Particles and Methods of Making and Using Same, WO99/19515. Additional techniques for generating biochemical data are described in U.S. Pat. Nos. 5,736,330, 5,981,180, 6,046,807, and 6,057,107. The disclosures of the preceding patent references are incorporated by reference herein.

This technology allows the simultaneous determination of the concentrations of multiple biochemicals in a single sample of blood or other biological fluids. In this application, this technology will be referred to as the “Luminex” technology, and the profile of concentrations of biochemicals derived is referred to as a Multi-Analyte Profile (MAP). Conventional technologies are slow, require excessive patient blood, and are prohibitively expensive.

In this application, the term “database” will be used interchangeably with “electronic database.” Other terms, which can be equivalently used for “database,” include “automated information retrieval system,” “computer readable database,” or “database accessible by a computer.” The term “database” does not refer to conventional medical records as, for example, kept in a doctor's office, hospital, or health maintenance organization even if in electronically searchable form.

The database created by this effort is the largest and most comprehensive repository of information about the complex biochemical processes underlying health and disease. It is expected that the present invention will enable the detection of cancer years earlier than is now possible with conventional technologies. Heart disease and diabetes are predicted in time to allow pre-symptomatic intervention. Ultimately, the fundamental defect and the complete characterization of every disease is identified by this invention.

To understand the importance of the integrative database created by this invention, it is helpful if an analogy is drawn between a sailor using celestial navigation to pinpoint his position on Earth, and our attempts to diagnose a medical problem. In both cases, accuracy is increased when more coordinates are considered in the determination. A navigator is most precise when multiple sextant readings of the sun, moon, planets, and stars all contribute to his estimate of position. A single sighting taken at noon is dangerously susceptible to error from many possible sources. Even in the technologically advanced Global Positioning System, the highest accuracy involves readings from the largest number of satellites. Similarly, since the inventors believe that evidence of every biochemical event influencing sickness or health is detectable in the blood, the more of these events one analyzes and understands, the more accurate is one's diagnosis of incipient or active disease. As both the Luminex technology and the database of this invention evolve, one moves closer and closer to absolute precision in medical diagnosis. This precision may be delivered rapidly and at low cost.

In the creation of the database of the present invention, many types of test samples can be used. Preferred test samples comprise biological fluids, mixtures, or preparations thereof. More preferably, the one or more test samples comprise blood samples, mixtures, or preparations thereof. As stated elsewhere in this disclosure, preferred reagents bound to the microspheres comprises a small molecule, natural product, synthetic polymer, peptide, polypeptide, polysaccharide, lipid, nucleic acid, or combinations thereof. In turn, the predetermined analyte comprises a drug, hormone, antigen, antibody, protein, enzyme, DNA, RNA, or combinations thereof.

In performing the methods of the present invention, one may find it useful to add one or more supplemental reagents to assist, enhance, or facilitate the generation of biochemical data. Such supplemental reagents may comprise a substrate, antibody, affinity reagent, label, or combinations thereof. One of ordinary skill in the art may also find that there is some advantage to performing certain additional steps. Hence, one might choose to further filter the exposed microspheres from the one or more test mixtures prior to passing the filtered microspheres through the flow analyzer.

In general, the term “biochemical data” is broadly meant to capture a wide range of information of potential interest to medical investigators, but this term includes at least the presence, absence, or quantity of predetermined analyte present in the one or more test samples.

The underlying premise of the invention is the ability to obtain biochemical data on a large number of analytes and on a broad scale. Hence, the biochemical data preferably includes data concerning 20 or more predetermined analytes, more preferably, 100 or more predetermined analytes, and, most preferably, 300 or more predetermined analytes.

As discussed elsewhere in this disclosure, at least some or all of the subjects in a particular pool of subjects enjoy relatively good health. Yet in others some or all of the subjects suffer from relatively poor health. Clearly, a mixture of healthy or “normal” subjects and subjects in poor health will participate in the creation of the database of the present invention.

In specific embodiments of the invention, some or all of the subjects in a particular pool of subjects have been diagnosed with a disease or other pathological condition. In particular, some or all of the subjects have been diagnosed with a neoplastic, neurodegenerative, skeletal, muscular, connective tissue, skin, organ, metabolic, addictive, psychiatric disease, or combinations thereof.

Apart from obtaining or determining the subjects' medical histories, some or all of the subjects are subjected to a physical, medical, or psychiatric examination. Still others are requested to fill out a questionnaire.

The frequency by which test samples are obtained may vary. However, one or more test samples may be obtained from one or more subjects at least every month, quarter, biannually, or annually. Preferably, one or more test samples are obtained from one or more subjects annually over a period of at least three, five, seven, or nine years. Ideally, the examinations or questioning of the one or more subjects are conducted or performed, or their medical histories determined or obtained, annually over the same period.

In performing the correlation studies between the biochemical data generated and the medical histories, one preferably determines one or more changes in the biochemical data of the one or more subjects annually over the same period. One further determines one or more changes in the medical conditions or histories of the one or more subjects annually over the same period. Next, a relationship, if any, is determined between the one or more changes in the biochemical data and the one or more changes in the medical conditions or histories of the one or more subjects. In so doing, one finds that one or more changes in the biochemical data correlate with one or more changes in the medical conditions or histories of the one or more subjects. What is more, the analysis finds either that one or more changes in the biochemical data are predictive of one or more changes in the medical conditions or histories of the one or more subjects or that one or more changes in the biochemical data cause one or more changes in the medical conditions or histories of the one or more subjects.

Initially, about 1,000 subjects are involved in the generation of biochemical data. Over time, however, the pool of participating subjects grows to at least about 5,000, 10,000, 25,000, 50,000, 100,000 or more subjects.

4.1. Database of Patient “Normals”

The value of the information contained in the databse grows proportionately to the number and diversity of its participants. Representatives from every age, racial, socioeconomic, and geographic group in this country (initially) are included. Patients are admitted based on the above characteristics, and also on their likelihood of completing the five-year study. In addition to the Multi-Analyte Profile (MAP) Test Panel of at least about 200-300 biochemical markers analyzed from each patient, a thorough medical history is taken at least annually. Additional medical information is derived from approximately monthly surveys. In addition, information concerning the person's phenotype, such as height, weight, sex, race, hair and eye color is recorded.

In an optional expansion of the database, the blood sample is analyzed for genetic information which contributes to the diagnosis of disease state and prospective disease state of this invention.

4.2. Patient Management and Specimen/Data Collection

The preferred database is based on data collected in each of the fifty states. Health Centers have a diverse workforce of medically aware, minimally transient employees who are enthusiastic, dependable participants. Involvement of a well-respected Health Center also enhances credibility and brings the study “home” to each state. On average, 4,000 patients in each state are recruited for the study (more in California, less in North Dakota). A blood sample and a medical history are collected by two full-time employees in each office. All samples and identity-protected medical histories are forwarded to a laboratory for analysis and storage.

These studies also recruit patients with disease and assess the impact of new therapies. With samples from these patients, comparisons between healthy and diseased patient sera occur early in the study, and medically useful algorithms are compiled immediately thereafter. These algorithms are developed using advanced statistical analysis software and causal mathematics software, such as TETRAD. See, for example, Judea Pearl, Causality. Models, Reasoning, and Inference. Cambridge University Press (2000). The database is therefore available for commercial use within about 18 months of study initiation, offering dramatically improved diagnostic capability to patients tested by the Luminex technology.

At year one, the study has developed a “wellness” profile generated by the at least about 200-300 biochemical tests performed on blood samples from 200,000 volunteer participants. The database is expanded by the addition of many thousands of samples drawn from patients with known disease, which also are analyzed by Luminex technology. The uniquely low cost of performing the at least about 200-300 blood tests with this system provides the opportunity to test every possible sample that could contribute value to the database.

After year one, the database of the invention continues to grow, becoming an all-encompassing and increasingly powerful diagnostic platform. Some original participants have significantly different profiles in year two, allowing the biochemical manifestations of ongoing or incipient disease, or even a lifestyle change, to be recognized.

The correlations discovered in year one between a blood biochemistry profile and specific diseases allow development of the first diagnostic algorithm. Medical benefits derived from early profiling are compelling and apparent, and commercial testing begins nationwide.

4.3. Multi-Analyte Profiling (MAP), a Routine Diagnostic Tool

This simple low-cost procedure delivers sophisticated diagnostic information. A test of an individual's blood includes at least about 200-300 analyte MAP, and comparative analysis of patient results with the growing database. Profiling becomes an essential part of the routine annual check-up, offering all the common screening tests plus substantially more diagnostic information obtained by testing for hundreds of additional analytes and checking the results against the database.

The technology and the underlying worth of this diagnostic tool is first being proven in the U.S.A. Soon thereafter, the study database is expanded with the addition of population studies performed in the countries of Western Europe, Japan, and Australia/New Zealand. The analytical testing menu is not changed. However, the diagnostic algorithms developed for each country show differences due to the unique genetic, environmental, and cultural characteristics of the population. Many equatorial countries pose unique diagnostic problems that require specialized MAP's. For example, malaria, Lassa fever, and river blindness assays are not found on a MAP of the U.S. population, but are critically important in Africa.

The MAP of 200-300 analytes that initiates the study is growing into the thousands as the role of more blood biochemicals is defined. It is also important to note that the database is only “seeded” by the original 200,000 participants. As the MAP is expanded, each of the millions of annual tests becomes part of the database. The database even suggests effective therapeutic regimens based on a patient's MAP and the availability of advanced technology in a given country. For example, a diagnosis in the U.S. that would suggests organ transplantation may provide other options for a patient in Bolivia.

Genetic information derived from blood or biological fluids is optionally included in the database as supplemental information, which aids in deriving the correlation between changes in biological fluids and disease states and the development of disease states.

4.4. Database Security and the Internet

The medical and scientific value derived from the study resides in the integrative database. Access to the database is strictly controlled in order to prevent corruption or alteration of the data. Worldwide interaction with the database occurs over the Internet. Results of a patient's profile are sent over the “net” to a secure central server where they are evaluated against the database. A diagnostic report including suggestions for possible therapeutic modalities is then returned via the Internet to the lab where the MAP was performed.

It is important to understand another important advantage offered by the Internet. Every patient test that is evaluated by the database also expands the database. It quickly changes from a database built upon hundreds of thousands of patient profiles into one sifting information from hundreds of millions of patient profiles from around the world.

The ability to discern clinically relevant biochemical changes in the blood or other biological fluids is useful in other ways besides diagnosing disease. The extensive testing for safety and efficacy now required of pharmaceutical companies before the introduction of a new drug is covered by MAP studies. In the testing methods employed today, the biochemical alterations of a relatively few biochemical markers are studied. Side effects of drugs are detected by alterations in the hundreds of analytes in the database. The drug developer can detect such side effects with a simple clinical trial of 500 people, tested monthly for two years.

In addition, drugs that have already been approved are tested because the pharmaceutical companies want to learn more about the action of their drugs, to make them better and, again, to protect themselves from lawsuits stemming from side effects that could not be detected prior to the availability of the study's database.

The Luminex technology is extended to animal studies, developing MAPs for laboratory mice (used in biomedical research) and for veterinary applications.

4.5. Further Determinations Using Causal Methods and Related Considerations

It should be apparent that the present invention relates to a novel combination of large scale protein measurements and causal mathematics and statistics, which results in a series of mathematical models of human and animal biology. These models are created by measuring 20 to 10,000 proteins in blood, developing a profile of these proteins as they compare to observations of medical history, and, using causal methods, deriving a directed graph of protein interactions representative of normal and abnormal biological conditions.

Unlike classical statistics which are capable only of describing the probability that measurement “A” predicts disease “X”, causal methods as used in the present invention define a mathematical language for expressing that measurement “A” causes disease “X.” In doing so, equations are possible that describe a chain of protein interactions that eventually lead to disease. Further, the equations can incorporate the impact of intervening proteins or therapeutics that disrupt the chain and possibly “cure” the disease.

Hence, an important aspect of the invention relates to a method of predicting a disease in a subject comprising providing measurements of gene products in a sample obtained from the subject, applying causal mathematics and statistics, and determining causal interactions of gene products to predict the disease. In a particular embodiment of the method, the gene products comprise proteins. Moreover, the method can further comprise a comparison of at least one gene product to a control sample. Determining the causal interaction can involve deriving a graph. Ultimately, the causal mathematics leads to the derivation of an algorithm.

In the present invention, it is important to note that the causal mathematics permits comparison of subject and control samples. Accordingly, the application of causal methods leads to detection of early-stage disease. The method can utilize multiple measurements conducted at various times. Alternatively, a plurality of measurements can be made at one or a plurality of times. In a preferred embodiment, 20 or more measurements are made.

In a specific embodiment, the invention includes the derivation of an algorithm for predicting a disease in a subject comprising causal mathematics and statistics for evaluating information on protein levels in the subject and an output predictive of disease. Moreover, the mathematical relationship derived correlates the protein levels to disease. The mathematical relationship permits comparisons of the protein levels of test subjects with those of control subjects.

In yet another embodiment, the invention comprises a system for predicting a disease in a subject comprising a microprocessor, and an algorithm using causal mathematics and statistics for evaluating information on protein levels in the subject to provide an output, wherein the output is predictive of the disease. In addition, the system can further comprise a database of medical profiles for comparison with the subject.

In a still further embodiment, the invention comprises a method for developing a mathematical model predictive of disease comprising the steps of iterative application of an algorithm to a set of standard data to provide an output; and comparison of the output to a disease profile.

Yet a further embodiment of the invention is directed to a method for treating a disease comprising diagnosing a disease by the steps of providing measurements of gene products in a sample obtained from the subject, applying causal mathematics and statistics, and determining causal interactions of gene products to predict the disease; and applying a pharmacologic treatment specifically tailored to the disease.

5. Materials Methods and Examples for Obtaining Reagents and Target Analytes

U.S. Pat. Nos. 6,057,107; 6,046,807; 5,981,180; 5,802,327; 5,736,330 and PCT publications WO 00/50903; WO 99/58958; WO 99/58955; WO 99/57955; WO 99/52708; WO 99/37814; WO 99/36564; WO 99/19515; WO 98/59233; and WO 97/14028 provide useful background information pertaining to the invention. Each disclosure is incorporated by reference herein.

The Multi-Analyte Profile (MAP) Test Panel of the present invention comprises a collection of subsets of microspheres, the microspheres of each subset differing from those of another subset by at least one classification parameter (e.g., size, fluorescent color, non-fluorescent color, refraction index, magnetic property, density, etc.). Furthermore, the microspheres of each subset carry at least one distinct type of reagent.

It should be understood that the term “antibody” as used herein includes within its scope any of the various classes or sub-classes of immunoglobulins, e.g., IgG, IgA, IgM, or IgE derived from any of the animals conventionally or unconventionally used as a source of sera, such as sheep, rabbits, goats, or mice, to name a few. Antibody also encompasses monoclonal antibodies whether produced by cell fusion with immortalized cells or by recombinant techniques in eukaryotic or prokaryotic cells. Antibody also includes intact molecules or “fragments” of antibodies, monoclonal or polyclonal, the fragments being those which contain the binding region of the antibody, i.e., fragments devoid of the Fc portion (e.g., Fv, Fab, Fab′, F(ab′)₂ or fragments obtained by reductive cleavage of the disulfide bonds connecting the heavy chain components in the intact antibody, so long as they retain antigen binding capabilities).

The term “antigen” is understood to include both naturally antigenic species (for example, drugs, proteins, bacteria, bacterial fragments, cells, cell fragments, carbohydrates, nucleic acids, lipids, and viruses, to name a few) and haptens, which may be rendered antigenic under suitable conditions and recognized by antibodies or antibody fragments.

The present method is useful for the detection and analysis of a wide variety of analytes. The term “analyte” is meant to be construed broadly and includes “antigens,” “antibodies,” “enzymes,” “nucleic acids,” and the like. but is not solely limited to “antigens”. Many types of analytes are conceived, including, for example, environmental contaminant analytes, agricultural products, industrial chemicals, water treatment polymers, pharmaceutical drugs, drugs of abuse, and biological analytes, such as antigenic determinants of proteins, polysaccharides, glycoproteins, lipoproteins, nucleic acids, hormones, and parts of organisms, such as viruses, bacteria, fungi, parasites, plants and microbes.

The term “reagent” refers to the reaction partner or binding partner of an analyte. The molecular interactions between reagent and analyte are generally selective, preferably specific. Preferred analyte:reagent (or vice-versa) couples, however, include, but are not limited to, antigen:specific immunoglobulin; hormone:hormone receptor; nucleic acid strand:complementary polynucleotide strand; avidin:biotin; protein A:immunoglobulin; protein G:IgG immunoglobulins; enzyme: substrate; lectin: specific carbohydrate; drug:protein; small molecule:protein, and the like.

Known and unknown analytes, such as proteins, present in a clinical sample can be obtained by purification to serve as a reference material. Synthetic or recombinant peptides, polypeptides and proteins can also be prepared from the sequence information from any of a number of publicly accessible protein databases, including those available on the Internet. For example, such databases include PubMed, SwissProt, PIR, PRF, PDB, and translations from annotated coding regions in GenBank and RefSeq (http://www.ncbi.nlm.nih.gov/PubMed). Other Internet sites with protein databases, suitable for retrieving sequences of proteins or their fragments, include:

http://www.kazusa.or.jp/huge/;

http://alces.med.umn.edu/dbmotif.html;

http://www.harefield.nthames.nhs.uk/nhli/protein/other_sites.html;

http://www.biomed.man.ac.uk/ugrad/biomedical/calpage/sproject/alf/biodb.html;

http://sphinx.rug.ac.be:8080/other2D.html;

http://discover.nci.nih.gov/host/prot.html;

http://www.infobiogen.fr/services/dbcat/data/dbcat_PROT.html;

http:flwww.infobiogen.fr/services/deambulum/english/db4.html;

http://www.cybergenome.com/tools/databases.htm;

http://www.genome.ad.jp/manuscripts/GIW94/Poster/GIW94P06.html;

http://www2. links2go.com/topic/Protein_Databases;

http://www.biochemie.net/links/Databases/Protein/;

http://www.bioscience.org/urllists/protdb.htm; and

http://www.gcrdb.uthscsa.edu/help files/fast doc.html, among many others.

For the purpose of facilitating the selection of required reagents, the contents of these databases and updates thereof should be used advantageously.

Furthermore, one can easily make antibodies or binding pairs against any of these proteins. Also, antibodies against some of these proteins are readily available. For example, the publication on http://gbsl.freeservers.com provides more than 1900 monoclonal antibodies, including anti-idiotype, bispecific, human, chimeric, diabodies, single chain Fv, etc. The MSRS (Manufacturers' Specifications and Reference Synopsis) Primary Antibody Database is an online reference source that lists over 76,000 monoclonal and polyclonal primary antibodies. The URL is http://www.antibodies-probes.com.

Of course, one can order custom-made antibodies from various commercial manufacturers. The http://www.antibodyresource.com website provides an exhaustive list of companies making and/or selling such reagents: Bethyl Laboratories—(polyclonal, peptides); AbCam Ltd—(monoclonal, polyclonal); Advanced ChemTech—(polyclonal, peptides); AgriSera AB—(monoclonal, polyclonal, peptides); Anaspec—(polyclonal, peptides); Anawa Trading Company SA—(monoclonal, polyclonal, peptides); Antibody Solutions—(monoclonal, polyclonal, peptides)—in vitro production; Affiniti Research Products Ltd.—(UK)—(polyclonal, peptides); Affinity BioReagents, Inc.—(polyclonal); Alpha Diagnostics—(monoclonal, polyclonal, peptides); Antibodies Incorporated—(monoclonal, polyclonal); Aurora Biomolecules—(polyclonal, peptides); Aves Lab—(polyclonal)—chicken antibodies; B & K Universal, Ltd.—(monoclonal, peptides); Berkeley Antibody Company—(monoclonal, polyclonal); BIOCON, Inc.—(monoclonal, polyclonal)—in vitro; BioDiversa—(monoclonal, polyclonal, peptides); Biogenes—(monoclonal, polyclonal, peptides); Biogenesis—(monoclonal, polyclonal, peptides); Bio-Express—(monoclonal)—in vitro and IgG fragment production; Bioinvent International AB—(monoclonal)—human monoclonal antibodies; Bionostics, Inc.—(monoclonal, polyclonal); Bioquest—(monoclonal, polyclonal); BioSource International—(monoclonal, polyclonal); Bio-Synthesis—(monoclonal, polyclonal, peptides); Biotrend—(monoclonal, polyclonal, peptides); Biovendor—(monoclonal, polyclonal); Bioworld—(monoclonal, polyclonal, peptides); Babraham Technix—(monoclonal, polyclonal); Capralogics—(polyclonal); Cell Essentials—(monoclonal, polyclonal, peptides)—bioreactor production and antibody purification; Charles River Laboratories—(polyclonal); Charles River Laboratories SPAFAS—(polyclonal)—custom manufacturing of antibodies (antiserum or IgY); Cosmix—phage-display based services including custom mouse Fab antibodies; Covalab—(monoclonal, polyclonal, peptides)—chicken antibodies; Covance—(monoclonal, polyclonal); Custom Monoclonals International—(monoclonal); Diatec—(monoclonal); Fitzgerald Industries International, Inc.—(monoclonal, polyclonal); Flock Antibodies—(polyclonal)—chicken antibodies; Gallus Immunotech—(polyclonal)—chicken antibodies; Gallina Biotechnology—(polyclonal)—chicken antibodies; Geneka Biotechnology, Inc.—(polyclonal); Genemed Synthesis, Inc.—(monoclonal, polyclonal, peptides); Genosys—(polyclonal, peptides); Gramsch Laboratories—(monoclonal, polyclonal, peptides); Green Mountain Antibodies, Inc.—(monoclonal); Harlan Bioproducts—(monoclonal, polyclonal, peptides); ICN Biomedicals, Inc.—(polyclonal); Imgenex—(monoclonal, polyclonal); Immunechem—(polyclonal); Immunochem Diagnostics Technology, Inc.—(monoclonal, polyclonal); Immunosystem—(polyclonal)—chicken antibodies; Immunological Resource Center—(monoclonal, polyclonal)—in vitro production; ISL (Immune Systems Ltd)—(monoclonal, polyclonal, peptides); Lampire Biological Laboratories—(monoclonal, polyclonal); Lee Laboratories—(polyclonal); Morphosys—humanized antibodies; Maine Biotechnology Services, Inc.—(monoclonal, polyclonal, peptides); Mathison Immuno Scientific, Inc.—(monoclonal); Mediclone—(monoclonal); MicroPharm Ltd.—(polyclonal); Panigen—(monoclonal, polyclonal); ProtoPROBE, Inc.—(monoclonal, polyclonal, peptides)—recombinant single chain fragment variables (ScFv) and recombinant phage antibodies; Pocono—(monoclonal, polyclonal); QED Biosciences, Inc.—(monoclonal, polyclonal)—in vitro production, anti-idiotype and bifunctional antibody production; Quality Bioresources, Inc.—(polyclonal); Quality Controlled Biochemicals Corporation—(polyclonal, peptides); Research Genetics—(polyclonal, peptides); Terra Nova Biotechnology—(monoclonal, polyclonal)—in vitro; Rockland—(monoclonal, polyclonal, peptides)—tissue culture mAb production and DNA based immunizations (with vector construction); Southern Biotechnology Associates, Inc.—(monoclonal, polyclonal, peptides); Terra Nova Biotechnology—(monoclonal, polyclonal); Spring Valley Laboratories—(monoclonal, polyclonal, peptides); Washington Biotechnology, Inc.—(monoclonal, polyclonal, peptides); Yes Biotech Laboratories, Ltd.—(monoclonal); and Zymed company—(monoclonal, polyclonal, peptides) among many others.

5.1. Preparation of Host-Derived Antibodies Recognizing the Host's Disease

Circulating B lymphocytes derived from a neoplastic human host are cloned by fusion with immortalized human cell lines to provide hybridomas secreting monoclonal antibodies (MAbs) specific for a cell surface antigen of a neoplastic cell. Particularly, monoclonal antibodies specific for antigens of solid tumor cells, such as breast cancer cells or leukemic cells which are not found on normal cells of the same tissue type, are provided for use in diagnostics and therapy.

5.2. Preparation of Polyclonal Antibodies

Pathogen-free New Zealand white rabbits, weighing approximately 2-3 kg, are quarantined and acclimated in a pathogen-free facility prior to obtaining a preimmunization blood sample from each animal. One week after the pre-immunization bleed, a 1:1 dilution of an immunogenic enhancer comprising colloidal gold having an alkaline pH, mixed at a ratio of about 2:1, antigen solution to gold (Assay Research, Inc.) is mixed with 500 microgram of each peptide. The enhancer allows the peptide to act as the immunogenic molecule without conjugation to larger and more antigenic molecules such as BSA or KLH. For the first immunization, the peptide-adjuvant mixture is emulsified in Freund's complete adjuvant and injected subcutaneously into one rabbit. Two weeks later the peptide/enhancer mixture is emulsified in Freund's incomplete adjuvant and is injected again subcutaneously. Three days after this injection, five ml of blood is drawn through an ear vein and the resultant sera is tested for antibody titers. Approximately two weeks after the second injection, each rabbit is boosted with only the peptide/enhancer mixture and bled four days later. Subsequent injections, containing only the peptide enhancer mixture, and bleeds are performed once a month.

After a second injection of the antigen into a rabbits, a five ml blood sample is drawn and the serum tested for antibody titer. Typically, a two log dilution of the neat sera (i.e., 1:100 dilution to 1:10,000 dilution) does not decrease the signal generated. Although the antisera titers are high, the neat sera can not be used for further assay development due to rather high background color generation. Consequently, antisera is purified as described hereinafter.

Each polyclonal antiserum is purified by column chromatography using a mixed ion exchange resin (J. T. Baker, Inc., Phillipsburg, N.J.). The resin fractionates the serum into two major fractions: one fraction containing serum contaminants such as albumin and transferrin and the other fraction containing a highly enriched immunoglobulin fraction. The resin-bound antibody is eluted from the column using a linear gradient of 0 to 0.75 M NaCl in 25 mM MES (2-N-Morpholinoethanesulfonic acid) (pH 5.6 without NaCl, pH 7.0 at 0.75 M NaCl). Five ml fractions are collected and analyzed for protein content (absorption at 280 nm). The presence of specific antibodies is tested by a direct enzyme immunoassay (EIA). Rabbit antibodies are detected by an alkaline phosphatase-conjugated goat anti-rabbit antibody. Those fractions which result in a signal-to-noise ratio of five or more are pooled and dialyzed against PBS. The resultant pooled aliquots serve as the antibody solution for further use.

Alternatively, rabbit polyclonal antibodies are prepared by immunizing a rabbit with polyacrylamide gel material containing affinity-purified protein of interest. The IgG fraction is isolated from the obtained antiserum and absorbed by passage through columns with immobilized human protein.

5.3. Production of Monoclonal Antibodies

While cell fusion, cloning and propagation of hybridomas can be performed according to standard procedures, below are provided the specific details.

Mice of the BALB/c strain are immunized by giving three intraperitoneal injections with 5 microgram of antigen with 3 week intervals. 8-10 days after the last injection, serum is tested in both ELISA and Western blotting for reactivity against the immunogen. When positive reaction is detected, a final booster injection of 10-15 microgram of immunogen is given intraperitoneally.

The spleen and peripheral lymph nodes from an immunized BALB/c mouse are mechanically disrupted, and homogeneous cell suspensions are prepared in serum-free medium.

Myeloma cells in logarithmic phase of growth are resuspended in serum-free medium and readied for fusion with BALB/c spleen lymphocytes. The spleen and lymph node lymphocytes and myeloma cells are mixed in a ratio of 1:1.25 and 1:2, respectively. Cells were fused by dropwise addition of 50% (wt/vol) polyethylene glycol 4000 (PEG) at 37° C. at about 5 ml to 10⁸ and 1 ml to 4.5×10⁷ for the spleen and lymph node lymphocytes, respectively. The fusion is stopped by gentle addition of serum-free medium. After centrifugation, the supernatant is removed and the cells are washed once in serum containing medium. The cells are then carefully resuspended in hypoxanthine-aminopterin-thymidine (HAT)-containing medium. The fused cells at an amount of approximately 7×10⁵ cells/well (spleen fusion) and 5×10⁵ cells/well (lymph node fusion) are distributed in 50 microliter aliquots to wells of flat-bottomed microtiter plates containing 150 microliter of selection medium. The cells are incubated at 37° C. in 5% CO² in a humidified incubator. The selection medium is renewed after a week or when needed. The wells are inspected for hybridoma growth. When vigorous growth and change of color to yellow is observed, supernatants are removed for screening for antibodies reacting with immunogen by an ELISA method. 10-14 days after fusion, HAT medium is replaced by HT medium and later, e.g., after 10 days, by regular medium. ELISA-positive wells are transferred into cups of 24-well plates and then to small 25 cm culture flasks. ELISA-positive hybrid cells are frozen in liquid nitrogen as early as possible. Hybridomas from ELISA positive wells are cloned by limited dilution.

Antibodies are then purified as follows: The Protein G Sepharose 4 FF column is opened by removing the top cap first. This will avoid air bubbles being drawn into the gel. The 20% ethanol storage solution is poured off and the Protein G Sepharose 4 FF column is equilibrated by filling it to the top with Binding Buffer (˜30 ml) whereafter the column is allowed to drain. The column will stop flowing automatically as the meniscus reaches the top frit, preventing the column from drying out. The culture supernatants are centrifuged, filtered and 50-150 ml of the prepared sample is applied and allowed to absorb into the gel. Unbound proteins are washed away by filling the columns to the top with Binding Buffer and the buffer is allowed to pass through the column, eluting unbound materials. The bound IgG is eluted by filling the column with Elution Buffer on the column. One ml fractions of eluted antibodies are collected in minisorb tubes containing neutralizing buffer, and the purity of the elution fractions is checked on a 8-25% gradient gel employing Phast gel System (Pharmacia) followed by silver staining. Isotyping of obtained monoclonal antibodies is achieved by Mouse Typer Sub-Isotyping kit (Bio-Rad).

5.4. Coupling of Reagents to Microspheres to Provide MAP Test Panel

Twenty or more reagents, each intended for a different analyte suspected of being present in a test sample, are coupled to uniformly sized microspheres according to the literature. Each of the twenty or more reagents is coupled to a specific subset of microspheres, which are dyed with two types of fluorescent materials, such that each subset exhibits a characteristic fluorescence signature. The characteristic fluorescence signature allows a flow analyzer to distinguish the members of one subset from those of another. Twenty or more unique subsets of microspheres are prepared, each according to methods similar to those described, e.g., in PCT Application Number US98/21562. The twenty or more subsets of microspheres, each subset targeted to a different predetermined analyte, are combined to provide a MAP Test Panel of the present invention. Kits are also prepared comprising the MAP Test Panel and associated buffers, vials and supplemental reagents.

5.5. Testing Samples Obtained from Volunteers

One or more test samples, typically, blood samples, are obtained from volunteers located nationwide. Their medical conditions are also evaluated and their medical histories obtained or determined. The test samples are exposed to the MAP Test Panel and the results (i.e., biochemical data generated) are recorded using a flow analyzer. Biochemical data from thousands of volunteers are compiled in a database, which can be cross-checked with the identities and accompanying medical conditions or histories of the individuals from which the biochemical entries originated.

Test samples are periodically (e.g., biannually) withdrawn from the volunteers over a period of five years. Each time the health, condition and medical records of each volunteer are updated.

Careful examination of the information presented in the database, even after a short period of 18 months, reveals relationships between features of the biochemical data and the relative health or medical condition of the subjects. Indeed, the development of pathological conditions is foretold by the biochemical data generated in advance of a formal diagnosis or of the appearance of clinical disease.

Similarly, a separate group of subjects are followed over the course of drug administration or experimental therapy, to obtain direct information about the effects of same on protein expression levels and their consequences on the health, recovery, or the occurrence of unwanted complications.

It should be apparent to those of ordinary skill that the present invention is not limited by the examples and preferred embodiments described in this disclosure, which simply illustrate the invention. Other embodiments may come to mind, which fall within the scope and spirit of the invention, which is limited solely by the claims that follow.

1.-7. (Canceled)

8. A method of assessing a subject's health or medical condition comprising:
(a) providing one or more test samples obtained from a subject;
(b) exposing the one or more test samples to a Multi-Analyte Profile (MAP) Test 25 Panel comprising 20 or more subsets of microspheres, the microspheres of one subset being distinguishable from those of another subset and harboring at least one reagent designed to interact selectively, if not specifically, with a predetermined analyte, which interaction generates biochemical data concerning the predetermined analyte;
(c) gathering the biochemical data, if any, generated from the exposure;
(d) comparing the biochemical data generated from the one or more samples obtained from the subject with accumulated biochemical data generated from test samples taken periodically from at least about 1,000 individuals over a given time interval, which accumulated biochemical data provide a relationship between one or more predetermined analytes and the health or medical condition of a plurality of individuals whose accumulated biochemical data share similar features; and
(e) assessing the health or medical condition of the subject based, at least in part, on the results of the comparison.

9. The method of claim 8 in which the given time interval is as long as about three years.

10. The method of claim 8 in which the given time interval is as long as about five years.

11. A method of creating a database containing biochemical data from at least about 1,000 subjects, comprising:
(a) providing one or more test samples obtained from one or more subjects;
(b) exposing a Multi-Analyte Profile (MAP) Test Panel to at least a portion of the one or more test samples to provide one or more test mixtures, the MAP Test Panel comprising 20 or more subsets of microspheres, the microspheres of one subset being distinguishable from those of another subset and harboring at least one reagent designed to interact selectively, if not specifically, with, and to generate biochemical data concerning, a predetermined analyte;
(c) optionally, adding one or more supplemental reagents to the one or more test mixtures to further the generation of the biochemical data;
(d) passing the exposed microspheres of the one or more test mixtures through a flow 20 analyzer to extract the biochemical data generated;
(e) compiling the biochemical data into a database, which permits retrieval of the biochemical data at least according to the identities or medical histories of the one or more subjects from which the one or more test samples were obtained; and
(f) repeating some or all of the foregoing steps until biochemical data from at least 25 about 1,000 subjects are compiled into the database.

12. The method of claim 11 in which the one or more test samples comprise biological fluids, mixtures, or preparations thereof.

13. The method of claim 11 in which the one or more test samples comprise blood samples, mixtures, or preparations thereof.

14. The method of claim 11 in which the MAP Test Panel comprises 50 or more, 75 or more, 100 or more, 200 or more, or 300 or more subsets of microspheres.

15. The method of claim 11 in which the microspheres of one subset are distinguishable from those of another subset by their characteristic fluorescence signatures.

16. The method of claim 11 in which the at least one reagent comprises a small molecule, natural product, synthetic polymer, peptide, polypeptide, polysaccharide, lipid, nucleic acid, or combinations thereof.

17. The method of claim 11 in which the predetermined analyte comprises a drug, hormone, antigen, antibody, protein, enzyme, DNA, RNA, or combinations thereof.

18. The method of claim 11 in which the one or more supplemental reagents comprises a substrate, antibody, affinity reagent, label, or combinations thereof.

19. The method of claim 11 which further comprises filtering the exposed microspheres from the one or more test mixtures prior to passing the filtered microspheres through the flow analyzer.

20. The method of claim 11 in which the biochemical data includes the presence, absence, or quantity of predetermined analyte present in the one or more test samples.

21. The method of claim 11 in which the biochemical data includes data concerning or more predetermined analytes.

22. The method of claim 11 in which some or all of the subjects enjoy relatively good health.

23. The method of claim 11 in which some or all of the subjects suffer from relatively poor health.

24. The method of claim 11 in which some or all of the subjects have been diagnosed with a disease or other pathological condition.

25. The method of claim 11 in which some or all of the subjects have been diagnosed with a neoplastic, neurodegenerative, skeletal, muscular, connective tissue, skin, organ, metabolic, addictive, psychiatric disease, or combinations thereof.

26. The method of claim 11 in which some or all of the subjects are subjected to a physical, medical, or psychiatric examination.

27. The method of claim 11 in which the medical histories of some or all of the subjects are determined or obtained.

28. The method of claim 11 in which some or all of the subjects are requested to fill out a questionnaire.

29. The method of claim 11 in which one or more test samples are obtained from one or more subjects at least every month, quarter, biannually, or annually.

30. The method of claim 11 in which one or more test samples are obtained from one or more subjects annually over a period of at least three, five, seven, or nine years.

31. The method of claim 30 in which the examinations or questioning of the one or more subjects are conducted or performed, or their medical histories determined or obtained, annually over the same period.

32. The method of claim 31 which further comprises determining one or more changes in the biochemical data of the one or more subjects annually over the same period.

33. The method of claim 32 which further comprises determining one or more changes in the medical conditions or histories of the one or more subjects annually over the same period.

34. The method of claim 33 which further comprises determining the relationship, if any, between the one or more changes in the biochemical data and the one or more changes in the medical conditions or histories of the one or more subjects.

35. The method of claim 34 in which the determining step finds that one or more changes in the biochemical data correlate with one or more changes in the medical conditions or histories of the one or more subjects.

36. The method of claim 34 in which the determining step finds that one or more changes in the biochemical data are predictive of one or more changes in the medical conditions or histories of the one or more subjects.

37. The method of claim 34 in which the determining step finds that one or more changes in the biochemical data cause one or more changes in the medical conditions or histories of the one or more subjects.

38. The method of claim 11 in which biochemical data from at least about 5,000, 10,000, 25,000, 50,000, or 100,000 subjects are compiled into the database.
